(19) United States
(12) Patent Application Publication
(10) Pub. No.: US 2022/0387974 A1
(43) Pub. Date: Dec. 8, 2022

(54) CORE-SHELL STRUCTURE SUPPORTED CATALYST AND PREPARATION METHOD AND USE THEREOF

(71) Applicant: Zhejiang Shuren College(Zhejiang Shuren University), Shaoxing City (CN)

(72) Inventors: Chao SHEN, Shaoxing City (CN); Nabo SUN, Shaoxing City (CN); Kai ZHENG, Shaoxing City (CN); Hao XU, Shaoxing City (CN); Huizhen WU, Shaoxing City (CN); Jun QIAO, Shaoxing City (CN)

(21) Appl. No.: 17/467,777

(22) Filed: Sep. 7, 2021

(30) Foreign Application Priority Data
May 31, 2021 (CN) 202110600290.3

(51) Int. Cl. B01J 23/42; B01J 35/02; B01J 35/00

(52) U.S. Cl. CPC B01J 23/42; B01J 35/023; B01J 35/008; B82Y 30/00

(57) ABSTRACT

A core-shell structure supported catalyst and a preparation method and use thereof are disclosed. The core-shell structure supported catalyst includes a core-shell structure carrier and platinum supported on the surface of the core-shell structure carrier, wherein the core-shell structure carrier includes a ferroferric oxide nanoparticle core and a nitrogen-doped carbon shell, and a molar ratio of the ferroferric oxide nanoparticle core to platinum is 1:(0.03-0.3).

### CROSS REFERENCE TO RELATED APPLICATION

The patent application claims the benefit and priority of Chinese Patent Application No. 202110600290.3 filed on May 31, 2021, the disclosure of which is incorporated by reference herein in its entirety as part of the present application.

### TECHNICAL FIELD

The present disclosure relates to the technical field of catalytic synthesis, and specifically, relates to a core-shell structure supported catalyst and a preparation method and use thereof.

### BACKGROUND ART

Lorlatinib is a new drug for the treatment of non-small cell lung cancer, developed by Pfizer Pharmaceuticals Co., Ltd. It is specifically used for the treatment of anaplastic lymphoma kinase (ALK) positive metastatic non-small cell lung cancer. As the third generation ALK inhibitor, lorlatinib is approved for the treatment of lung cancer patients with ALK, especially patients whose disease continue to worsen after receiving crizotinib or at least one other ALK inhibitor treatment, or patients whose disease continue to worsen after receiving alectinib or ceritinib as the first ALK inhibitor treatment. Therefore, lorlatinib has the potential to significantly prolong survival of lung cancer patients who have developed resistance after receiving other ALK inhibitor treatment. Lorlatinib has the structure formula as shown below:

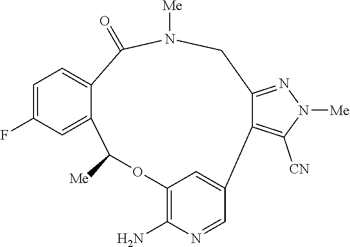 [SMILES: C[C@@H]1Oc2cc(cnc2N)-c2c(nn(C)c2C#N)CN(C)C(=O)c2ccc(F)cc21]

2-amino-5-fluorobenzoic acid is a key intermediate for the preparation of lorlatinib. At present, 2-amino-5-fluorobenzoic acid is mainly prepared by a method comprising using 5-fluoro-2-nitrobenzoic acid as a raw material, and subjecting 2-amino-5-fluorobenzoic acid to a palladium carbon hydrogenation reduction. The method has disadvantage that the reaction system is prone to side reactions of defluorination, which affects the purity and yield of the product. The purity of 2-amino-5-fluorobenzoic acid in the art is mostly 95.5-98.2%, and the yield is 76-93%.

### SUMMARY

In view of this, the present disclosure provides a core-shell structure supported catalyst and a preparation method and use thereof. Under the condition that the core-shell structure supported catalyst provided by the present disclosure is used to catalyze the preparation of 2-amino-5-fluorobenzoic acid, it could avoid the side reaction of defluorination, improving the purity and yield of the reduction reaction product.

In order to solve the above technical problem, the present disclosure provides a core-shell structure supported catalyst, comprising a core-shell structure carrier and platinum supported on the surface of the core-shell structure carrier, wherein the core-shell structure carrier comprises a ferroferric oxide nanoparticle core and a nitrogen-doped carbon shell, and a molar ratio of the ferroferric oxide nanoparticle core to platinum is in the range of 1:(0.03-0.3).

In some embodiments, the core-shell structure supported catalyst has a particle size of 20-100 nm.

In some embodiments, a mass ratio of the ferroferric oxide nanoparticle core to the nitrogen-doped carbon shell is in the range of (50-200):1;

a mass percentage of carbon in the nitrogen-doped carbon shell is in the range of 0.1-10%; and

a mass percentage of nitrogen in the nitrogen-doped carbon shell is in the range of 0.02-5%.

The present disclosure also provides a method for preparing the core-shell structure supported catalyst described in the above technical solution, comprising:

dispersing ferroferric oxide nanoparticles and chitosan in a first solvent, and subjecting the resulting dispersed mixture to a carbonization reaction, to obtain a core-shell structure carrier; and

mixing the core-shell structure carrier, a platinum compound, a reducing agent, and a second solvent, and subjecting the resulting mixed solution to a reduction reaction, to obtain the core-shell structure supported catalyst.

In some embodiments, the platinum compound is one selected from the group consisting of platinum chloride, chloroplatinic acid hexahydrate, and sodium hexachloroplatinate;

the reducing agent is one selected from the group consisting of ascorbic acid, hydrazine hydrate, and sodium borohydride.

In some embodiments, the first solvent is an aqueous acetic acid solution;

the carbonization reaction is performed at a temperature of 180-200° C. for 10-12 h.

In some embodiments, the second solvent is one selected from the group consisting of an aqueous ethanol solution, water, and an aqueous methanol solution;

the reduction reaction is performed at a temperature of 60-100° C. for 2-3 h.

In some embodiments, the ferroferric oxide nanoparticles are prepared by a method comprising:

mixing an iron compound, ethylene glycol, polyvinylpyrrolidone, and sodium acetate, and subjecting the resulting mixture to a solvothermal reaction, to obtain the ferroferric oxide nanoparticles.

The present disclosure also provides use of the core-shell structure supported catalyst described in the above technical solution or the core-shell structure supported catalyst prepared by the method described in the above technical solution in the preparation of 2-amino-5-fluorobenzoic acid.

In some embodiments, the preparation of 2-amino-5-fluorobenzoic acid comprises:

mixing 5-fluoro-2-nitrobenzoic acid, water and the core-shell structure supported catalyst, to obtain a reaction solution, wherein a mass ratio of 5-fluoro-2-nitrobenzoic acid to the core-shell structure supported catalyst is in the range of (5-20):1; and

adding ammonium formate into the reaction solution, and subjecting the resulting mixture to a reduction reaction, to obtain 2-amino-5-fluorobenzoic acid.

The present disclosure provides a core-shell structure supported catalyst, comprising a core-shell structure carrier and platinum supported on the surface of the core-shell structure carrier, wherein the core-shell structure carrier comprises a ferroferric oxide nanoparticle core and a nitrogen-doped carbon shell, and a molar ratio of the ferroferric oxide nanoparticle core to platinum is in the range of 1:(0.03-0.3). In the present disclosure, platinum is evenly dispersed on the surface of the core-shell structure carrier, which avoids the agglomeration of platinum, and at the same time, limits the loading amount of platinum in the catalyst, making the catalytic activity of the catalyst maintain in an appropriate range (both could catalyze reduction reaction, but not enough to defluorinate), and avoiding the side reaction of defluorination in the preparation of 2-amino-5-fluorobenzoic acid, thereby improving the purity and yield of the product.

### BRIEF DESCRIPTION OF THE DRAWINGS

FIG. 1 shows a flow chart of the preparation of the core-shell structure supported catalyst, wherein ferric chloride is used as an iron compound, and chloroplatinic acid hexahydrate is used as a platinum compound, and sodium borohydride is used as a reducing agent.

FIG. 2 shows a transmission electron microscope image of the core-shell structure supported catalyst prepared in Example 1.

FIG. 3 is graph showing a magnetic VSM curve of the core-shell structure carrier prepared in Example 1.

FIG. 4 is a diagram showing the core-shell structure supported catalyst prepared in Example 1 before and after magnetic separation.

### DETAILED DESCRIPTION OF THE EMBODIMENTS

The present disclosure provides a core-shell structure supported catalyst, comprising a core-shell structure carrier and platinum supported on the surface of the core-shell structure carrier, wherein the core-shell structure carrier comprises a ferroferric oxide nanoparticle core and a nitrogen-doped carbon shell.

In the present disclosure, a molar ratio of the ferroferric oxide nanoparticle core to platinum is in the range of 1:(0.03-0.3), and preferably 1:(0.08-0.2). According to the present disclosure, in some embodiments, a mass percentage of carbon in the nitrogen-doped carbon shell is in the range of 0.1-10%, and more preferably 0.5-2%; and a mass percentage of nitrogen in the nitrogen-doped carbon shell is in the range of 0.02-5%, and more preferably 0.1-2%. According to the present disclosure, in some embodiments, the ferroferric oxide nanoparticles have a partical size of 20-100 nm, and more preferably 50-80 nm. According to the present disclosure, in some embodiments, a mass ratio of the ferroferric oxide nanoparticle core to the nitrogen-doped carbon shell is in the range of (50-200):1, and more preferably (100-120):1.

In the present disclosure, platinum combines with nitrogen on the surface of the nitrogen-doped carbon shell in the form of coordination bond, and is evenly dispersed on the surface of the nitrogen-doped carbon shell, which avoids agglomeration. According to the present disclosure, in some embodiments, the core-shell structure supported catalyst has a particle size of 20-100 nm, and more preferably 50-80 nm. In the present disclosure, platinum is evenly dispersed on the surface of the nitrogen-doped carbon shell, which makes the catalytic activity of the core-shell structure supported catalyst reach the range that could catalyze the reduction of 5-fluoro-2-nitrobenzoic acid without the side reaction of defluorination. At the same time, the present disclosure further ensures the activity of the core-shell structure supported catalyst by limiting the particle size of the catalyst within the above range.

In the present disclosure, in the core-shell structure supported catalyst, the ferroferric oxide nanoparticles are used as the core; since they have magnetic properties and are easy to recycle, and thus could be recycled repeatedly.

The present disclosure also provides a method for preparing the core-shell structure supported catalyst in the above technical solution, comprising:

dispersing ferroferric oxide nanoparticles and chitosan in a first solvent, and subjecting the resulting dispersed mixture to a carbonization reaction, to obtain a core-shell structure carrier; and

mixing the core-shell structure carrier, a platinum compound, a reducing agent and a second solvent, and subjecting the resulting mixed solution to a reduction reaction, to obtain the core-shell structure supported catalyst.

In the present disclosure, ferroferric oxide nanoparticles and chitosan are dispersed in a first solvent, and the resulting dispersed mixture is subjected to a carbonization reaction, to obtain a core-shell structure carrier. In the present disclosure, the ferroferric oxide nanoparticles are prepared by a method comprising:

mixing an iron compound, ethylene glycol, polyvinylpyrrolidone, and sodium acetate, and subjecting the resulting mixture to a solvothermal reaction, to obtain the ferroferric oxide nanoparticles.

In the present disclosure, an iron compound, ethylene glycol, polyvinylpyrrolidone and sodium acetate are mixed, and the resulting mixture is subjected to a solvothermal reaction, to obtain the ferroferric oxide nanoparticles. According to the present disclosure, in some embodiments, the iron compound is one selected from the group consisting of ferric chloride, ferric chloride hexahydrate and ferrous chloride tetrahydrate, and more preferably ferric chloride hexahydrate. According to the present disclosure, in some embodiments, a ratio of the mass of the iron compound to the volume of ethylene glycol is in the range of 1 g:(30-40) mL, and more preferably 1 g:(33-35) mL; and a mass ratio of the iron compound, polyvinylpyrrolidone and sodium acetate is in the range of 1:(1.5-2.5):(3.5-4.5), and more preferably 1:(2-2.3):(3.8-4). In the present disclosure, polyvinylpyrrolidone acts as a dispersant, and sodium acetate acts as a stabilizer. Under the combined action of polyvinylpyrrolidone and sodium acetate, the prepared ferroferric oxide could be evenly dispersed in the suspension of the product, which avoids the agglomeration of ferroferric oxide, being conducive to the formation of core-shell structure.

According to the present disclosure, in some embodiments, the mixing is carried out under stirring conditions. In the present disclosure, there is no special limitation on the conditions of the rotation speed and time of the stirring, as long as the materials could be mixed to be uniform.

According to the present disclosure, in some embodiments, the solvothermal reaction is performed at a temperature of 160-200° C., and more preferably 180-190° C.; and the solvothermal reaction is performed for 6-10 h, and more preferably 8-9 h.

According to the present disclosure, in some embodiments, the method further comprises: after the solvothermal reaction, magnetically separating the resulting solvothermal reaction product, to obtain a solid; and washing and drying the solid in sequence, to obtain the ferroferric oxide nanoparticles. According to the present disclosure, in some embodiments, the washing is conducted by using ethanol as a solvent. According to the present disclosure, in some embodiments, the drying is performed by vacuum drying, and the vacuum drying is performed at a temperature of 50-70° C., and more preferably 60-65° C.; and the vacuum drying is performed for 11-13 h, and more preferably 12-12.5 h. In the present disclosure, there is no special limitation on the vacuum degree of the vacuum drying, as long as the vacuum conditions could be reached to avoid the oxidization of the ferroferric oxide.

According to the present disclosure, in some embodiments, dispersing ferroferric oxide nanoparticles and chitosan in a first solvent comprises:

dissolving chitosan in the first solvent, to obtain a chitosan solution; and

first mixing the chitosan solution and the ferroferric oxide nanoparticles.

In the present disclosure, chitosan is dissolved in the first solvent, to obtain a chitosan solution. According to the present disclosure, in some embodiments, the first solvent is an aqueous acetic acid solution, and a mass concentration of the aqueous acetic acid solution is in the range of 3-5%, and more preferably 3.5-4%. According to the present disclosure, in some embodiments, a ratio of the mass of chitosan to the volume of the first solvent is in the range of 2 g:(180-220) mL, and more preferably 2 g:(190-200) mL. According to the present disclosure, in some embodiments, the dissolving is carried out under ultrasound, and the ultrasound is performed at a ultrasound power of 200-500 W, and more preferably 250-300 W; the ultrasound is performed for 25-35 min, and more preferably 28-30 min.

In the present disclosure, after the chitosan solution is obtained, the chitosan solution is first mixed with ferroferric oxide nanoparticles. According to the present disclosure, in some embodiments, a mass ratio of the ferroferric oxide nanoparticles to chitosan is in the range of 0.8:(1.8-2.5), and more preferably 0.8:(2-2.2). According to the present disclosure, in some embodiments, the first mixing is carried out under ultrasound, and the ultrasound is performed at a ultrasonic power of 200-500 W, and more preferably 250-300 W; the ultrasound is performed for 25-35 min, and more preferably 28-30 min.

According to the present disclosure, in some embodiments, the method further comprises: after the mixing, adjusting the pH of the mixed product to 8-9 by using ammonia water. In the present disclosure, there is no special limitation on the concentration and amount of ammonia water, as long as the required pH value could be reached.

According to the present disclosure, in some embodiments, the carbonization reaction is carried out in an autoclave, and the autoclave is a stainless steel autoclave, and the lining of the autoclave is made of tetrafluoroethylene; the carbonization reaction is performed at a temperature of 180-200° C., and more preferably 185-190° C.; the carbonization reaction is performed for 10-12 h, and more preferably 10.5-11 h. In the present disclosure, under the above conditions, chitosan is carbonized on the surface of the ferroferric oxide nanoparticles to form nitrogen-doped carbon, forming a core-shell structure with the ferroferric oxide nanoparticles.

According to the present disclosure, in some embodiments, the method further comprises: after the carbonization reaction, subjecting the carbonization reaction product to a filtering, a washing with water, a washing with alcohol, and a vacuum drying in sequence, to obtain a core-shell structure carrier. In the present disclosure, there is no special limitation on the filtering, any conventional filtration method in the art may be used. In the present disclosure, the washing with water could remove the residual chitosan on the surface of the core-shell structure carrier. In the present disclosure, there is no special limitation on the amount of water used for the washing, as long as the purpose of removing the residual chitosan on the surface of the core-shell structure carrier could be achieved. According to the present disclosure, in some embodiments, the washing with alcohol is conducted for 1-5 times, and more preferably 2-3 times; the washing with alcohol is conducive to removing water on the surface of the core-shell structure carrier and drying. According to the present disclosure, in some embodiments, the vacuum drying is performed at a temperature of 55-65° C., and more preferably 60-63° C.; the vacuum drying is performed for 23-25 h, and more preferably 23.5-24 h.

In the present disclosure, after the core-shell structure carrier is obtained, a core-shell structure carrier, a platinum compound, a reducing agent and a second solvent are mixed, and the resulting mixture is subjected to a reduction reaction, to obtain the core-shell structure supported catalyst. According to the present disclosure, in some embodiments, the platinum compound is one selected from the group of platinum chloride, chloroplatinic acid hexahydrate, and sodium hexachloroplatinic acid, and more preferably chloroplatinic acid hexahydrate. According to the present disclosure, in some embodiments, the reducing agent is one selected from the group of ascorbic acid, hydrazine hydrate, and sodium borohydride, and more preferably sodium borohydride. According to the present disclosure, in some embodiments, the second solvent is one selected from the group of an aqueous ethanol solution, water and an aqueous methanol solution, and more preferably an aqueous ethanol solution; a mass concentration of the aqueous ethanol solution is in the range of 20-60%, and more preferably 30-40%; a mass concentration of the aqueous methanol solution is in the range of 20-60%, and more preferably 30-40%. According to the present disclosure, in some embodiments, a mass ratio of the ferroferric oxide nanoparticles to the platinum compound is in the range of 2:(0.13-0.18), and more preferably 2:(0.15-0.16). According to the present disclosure, in some embodiments, a mass ratio of the platinum compound to the reducing agent is in the range of 1:(1.8-2.2), and more preferably 1:2. According to the present disclosure, in some embodiments, a ratio of the mass of the core-shell structure carrier to the volume of the second solvent is in the range of 0.4 g:(35-45) mL, and more preferably 0.4 g:40 mL.

According to the present disclosure, in some embodiments, the mixing comprises:

dispersing the core-shell structure carrier in the second solvent, and second mixing the resulting mixture with the platinum compound and the reducing agent.

According to the present disclosure, in some embodiments, the dispersing is carried out by stirring, and the stirring is performed at a rotation speed of 200-500 r/min, and more preferably 280-350 r/min; the stirring is performed for 25-35 min, and more preferably 30 min. According to the present disclosure, in some embodiments, the second mixing is carried out by stirring. In the present disclosure, there is no special limitation on the rotation speed and time of the stirring, as long as the materials could be mixed to be uniform.

According to the present disclosure, in some embodiments, the reduction reaction is performed at a temperature of 60-100° C., and more preferably 60-80° C.; the reduction reaction is performed for 2-3 h, and more preferably 2.5-2.8 h. According to the present disclosure, in some embodiments, the reduction reaction is performed while stirring, and the stirring is performed at a rotation speed of 200-500 r/min, and more preferably 280-350 r/min.

According to the present disclosure, in some embodiments, the method further comprises: after the reduction reaction, subjecting the reduction reaction product to a magnetic separating, a washing, and a vacuum drying in sequence, to obtain the core-shell structure supported catalyst. According to the present disclosure, in some embodiments, the washing is conducted by using ethanol as a solvent, and the washing is conducted for 1-5 times, and more preferably 2-3 times. According to the present disclosure, in some embodiments, the vacuum drying is performed at a temperature of 60-95° C., and more preferably 65-85° C.; the vacuum drying is performed for 6-12 h, and more preferably 8-10 h.

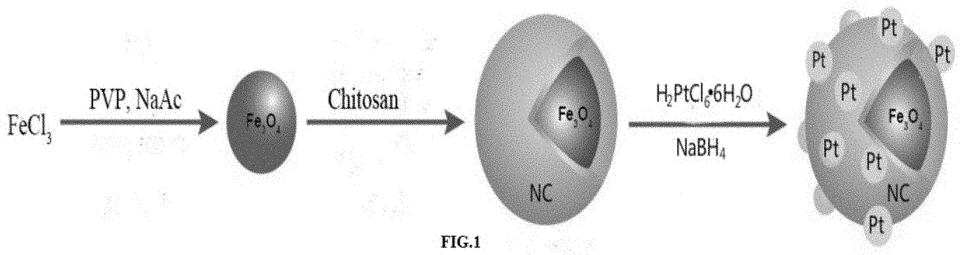

In the present disclosure, ferric chloride is used as the iron compound, and chloroplatinic acid hexahydrate is used as the platinum compound, and sodium borohydride is used as the reducing agent. The flow chart of the preparation of the core-shell structure supported catalyst is shown in FIG. 1.

The present disclosure also provides use of the core-shell structure supported catalyst described in the above technical solution or the core-shell structure supported catalyst prepared by the method described in the above technical solution in the preparation of 2-amino-5-fluorobenzoic acid. According to the present disclosure, in some embodiments, the preparation of 2-amino-5-fluorobenzoic acid comprises:

mixing 5-fluoro-2-nitrobenzoic acid, water and the core-shell structure supported catalyst, to obtain a reaction solution, wherein a mass ratio of 5-fluoro-2-nitrobenzoic acid to the core-shell structure supported catalyst is in the range of (5-20):1; and

adding ammonium formate into the reaction solution, and subjecting the resulting solution to a reduction reaction, to obtain 5-fluoro-2-aminobenzoic acid.

In the present disclosure, 5-fluoro-2-nitrobenzoic acid, water and the core-shell structure supported catalyst are mixed, to obtain a reaction solution, wherein a mass ratio of 5-fluoro-2-nitrobenzoic acid to the core-shell structure supported catalyst is in the range of (5-20):1, and preferably (10-15):1. According to the present disclosure, in some embodiments, a ratio of the mass of 5-fluoro-2-nitrobenzoic acid to the volume of water is in the range of 6.7 g:(55-65) mL, and more preferably 6.5 g:60 mL. According to the present disclosure, in some embodiments, the mixing is carried out by stirring. In the present disclosure, there is no special limitation on the rotation speed and time of the stirring, as long as the materials could be mixed to be uniform.

In the present disclosure, after the reaction solution is obtained, ammonium formate is added into the reaction solution, and subjected to a reduction reaction, to obtain 5-fluoro-2-aminobenzoic acid. According to the present disclosure, in some embodiments, the method further comprises: before the adding, heating the reaction solution to 80-90° C., and more preferably 85-88° C.

According to the present disclosure, in some embodiments, the reduction reaction is performed at a temperature of 85-95° C., and more preferably 90-94° C.; the reduction reaction is performed for 10-12 h, and more preferably 11-11.5 h.

According to the present disclosure, in some embodiments, the method further comprises: after the reduction reaction, adjusting a pH value of the reduction reaction product to 3-5, and filtering, to obtain a crude 2-amino-5-fluorobenzoic acid; and

beating the crude 2-amino-5-fluorobenzoic acid and an aqueous sulfuric acid solution with a pH of 3-5, to obtain 2-amino-5-fluorobenzoic acid.

In the present disclosure, the pH value of the reduction reaction product is adjusted to 3-5, and filtered, to obtain a crude 2-amino-5-fluorobenzoic acid. According to the present disclosure, in some embodiments, the pH value of the reduction reaction product is adjusted by an aqueous sulfuric acid solution. In the present disclosure, there is no special limitation on the concentration and amount of the aqueous sulfuric acid solution, as long as the required pH value could be reached. In the present disclosure, adjusting the pH value of the reduction reaction product to 3-5 is to utilize the reaction between sulfuric acid and ammonia gas to prevent 2-amino-5-fluorobenzoic acid in the product from reacting with ammonia gas generated to form a salt, thereby improving the purity and yield of 2-amino-5-fluorobenzoic acid. In the present disclosure, there is no special limitation on the filtering, any conventional operation in the art may be used.

In the present disclosure, after the crude 2-amino-5-fluorobenzoic acid is obtained, the crude 2-amino-5-fluorobenzoic acid and an aqueous sulfuric acid solution with a pH of 3-5 are beat, to obtain the 2-amino-5-fluorobenzoic acid. In the present disclosure, the beating could remove the ammonium formate remaining in the crude 2-amino-5-fluorobenzoic acid, which is conducive to further improving the purity of 2-amino-5-fluorobenzoic acid. According to the present disclosure, in some embodiments, the method further comprises suction filtration and rinsing after the beating. In the present disclosure, there is no special limitation on the suction filtration, any conventional operation in the art may be used. According to the present disclosure, in some embodiments, the rinsing is conducted by using an aqueous sulfuric acid solution with a pH of 3-5 as a solvent.

In the present disclosure, after the reduction reaction, the core-shell structure supported catalyst could be separated from the reaction product for recycling by magnetic separation. According to the present disclosure, in some embodiments, the resulting core-shell structure supported catalyst is recycled after the washing with water.

In the present disclosure, the catalytic reduction of 5-fluoro-2-nitrobenzoic acid to 2-amino-5-fluorobenzoic acid using the core-shell structure supported catalyst is conducted according to the reaction equation shown in formula I:

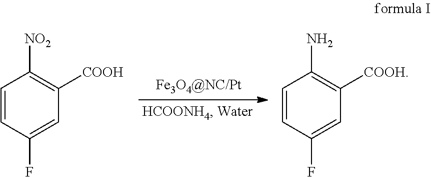 [SMILES: O=C(O)c1cc(F)ccc1[N+](=O)[O-] ; Nc1ccc(F)cc1C(=O)O]

In order to further illustrate the present disclosure, the technical solutions provided by the present disclosure will be described in detail below with reference with examples. It should not be understood as limiting the protection scope of the present disclosure.

Preparation of the Core-Shell Structure Supported Catalyst

### Example 1

3 g of FeCl₃.6H₂O, 2 g of polyvinylpyrrolidone, and 4 g of sodium acetate were dissolved in 60 mL of ethylene glycol, and the resulting mixed solution was subjected to a solvothermal reaction at 200° C. for 8 h, obtaining a solvothermal reaction product. The solvothermal reaction product was separated by magnetic separation, obtaining a solid. The solid was washed with ethanol. The washed solid was vacuum dried at 60° C. for 12 h, obtaining Fe₃O₄ nanoparticles with an average particle size of 70 nm.

2 g of chitosan was dissolved (the dissolving was performed at 300 W for 30 min) in 200 mL of an aqueous acetic acid solution with a mass concentration of 3%, obtaining a chitosan solution. The ferroferric oxide nanoparticles and the chitosan solution were mixed under ultrasound (the ultrasound was performed at 300 W for 30 min), obtaining a mixed product. The pH of the mixed product was adjusted to 8-9 by ammonia water. The resulting product after the adjustment was transferred to a stainless steel autoclave lined with tetrafluoroethylene, and subjected to a carbonization reaction (the carbonization reaction was performed at 200° C. for 12 h), obtaining a carbonization reaction product. After the carbonization reaction is completed, the carbonization reaction product was filtered, obtaining a solid. The solid was washed with water and alcohol (3 times). The washed product was vacuum dried at 60° C. for 24 h, obtaining a core-shell structure carrier, which was labeled as Fe₃O₄@NC.

0.4 g of the Fe₃O₄@NC was dispersed (while stirring at 400 r/min for 30 min) in 40 mL of aqueous ethanol solution with a mass concentration of 40%, and mixed with 0.15 g of chloroplatinic acid hexahydrate and 0.3 g of sodium borohydride to be uniform. The resulting mixture was subjected to a reduction reaction at 60° C. (while stirring at 400 r/min) for 2 h, obtaining a reduction reaction product. The reduction reaction product was separated by a magnet, obtaining a solid. The solid was washed 3 times with ethanol, and vacuum dried at 80° C. for 10 h, obtaining a core-shell structure supported catalyst, which was labeled as Fe₃O₄@NC/Pt.

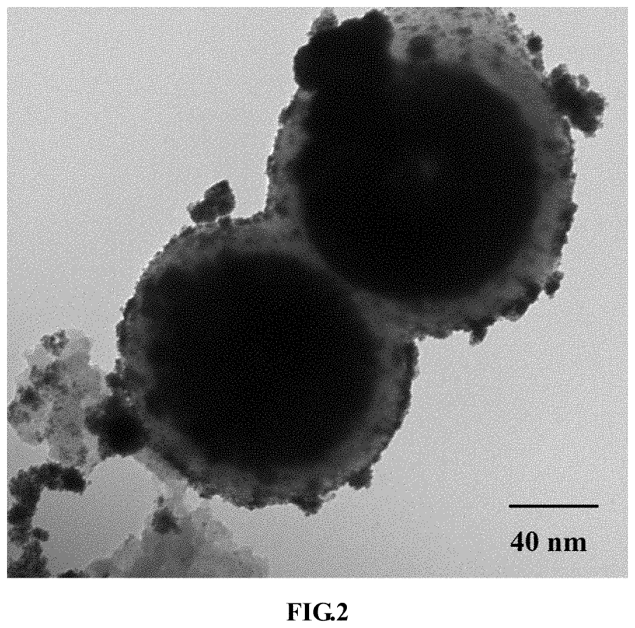

The Fe₃O₄@NC/Pt was detected by transmission electron microscopy, and the resulting transmission electron microscopy image is shown in FIG. 2. It can be seen from FIG. 2 that the core-shell structure supported catalyst provided by the present disclosure has a core-shell structure, and there are active components evenly dispersed on the shell surface. It can be seen from FIG. 2 that the core-shell structure supported catalyst has a particle size of 50-70 nm.

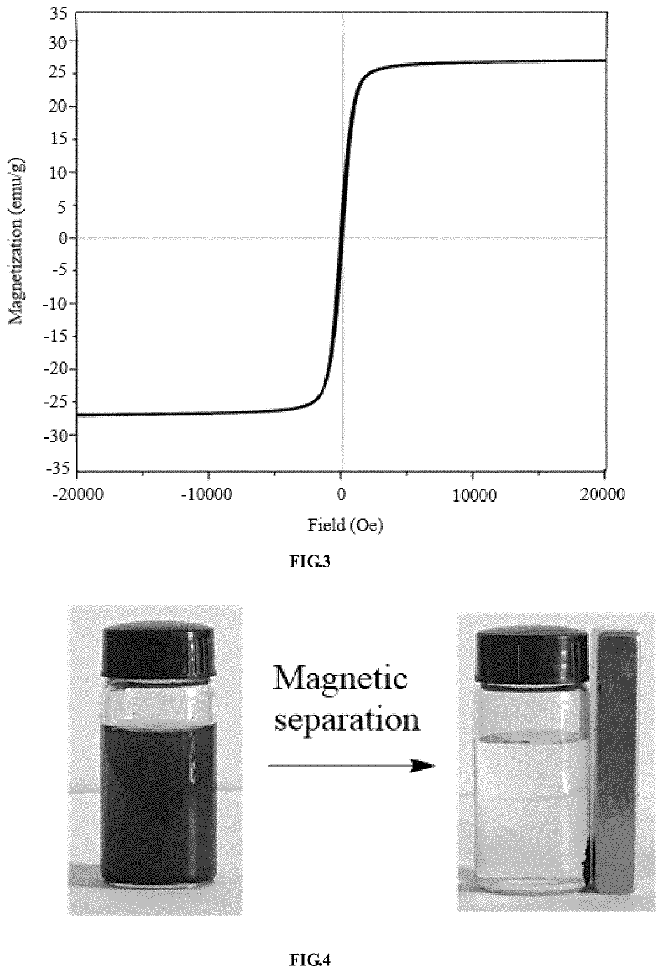

The magnetic detection of the Fe₃O₄@NC was performed with a vibrating sample magnetometer, and the resulting magnetic VSM curve after cycling is shown in FIG. 3. It can be seen from FIG. 3 that after many cycles, the magnetic properties of the core-shell structure carrier are not weakened, and effective magnetic separation can still be performed.

FIG. 4 is a diagram showing the core-shell structure supported catalyst before and after magnetic separation. It can be seen from FIG. 4 that the Fe₃O₄@NC can be separated well by magnetic separation with a magnet.

Catalytic reduction of 5-fluoro-2-nitrobenzoic acid using the Fe₃O₄@NC/Pt

### Example 2

6.7 g of 5-fluoro-2-nitrobenzoic acid, 0.6 g of the Fe₃O₄@NC/Pt and 60 mL of water were mixed, obtaining a reaction solution.

The reaction solution was heated to 85° C., and 9.6 g of ammonium formate was then added thereto, and the resulting mixture was further heated to 90° C. and subjected to a reduction reaction for 12 h, obtaining a reduction reaction product, The reduction reaction product was subjected to magnetic separation with a magnet to recycle the Fe₃O₄@NC/Pt. The remaining liquid after the magnetic separation was beat with 20 mL of an aqueous sulfuric acid solution with a pH of 3-5, and suction filtered, obtaining a filtrate. The filtrate was rinsed with the aqueous sulfuric acid solution with a pH of 3-5, obtaining 5-fluoro-2-aminobenzene.

### Example 3

In this example, 2-amino-5-fluorobenzoic acid is prepared by the method as described in Example 2, except that the Fe₃O₄@NC/Pt was used in an amount of 0.335 g, and the reduction reaction was performed at 85° C. for 10 h.

### Example 4

In this example, 2-amino-5-fluorobenzoic acid is prepared by the method as described in Example 2, except that the Fe₃O₄@NC/Pt was used in an amount of 1.34 g, and the reduction reaction was performed at 95° C. for 12 h.

The yield of 2-amino-5-fluorobenzoic acid was calculated according to the mass of 2-amino-5-fluorobenzoic acid prepared in Examples 2-4, and the results are listed in Table 1. The purity of the 2-amino-5-fluorobenzoic acid prepared in Examples 2-4 were tested by high performance liquid chromatography, and the results are listed in Table 1.

TABLE 1
The yield of 2-amino-5-fluorobenzoic acid prepared in Examples 2-4

| | Mass of | Yield of | Purity of |
|---|---|---|---|
| | 2-amino-5- | 2-amino-5- | 2-amino-5- |
| Examples | fluorobenzoic | fluorobenzoic | fluorobenzoic |
| No. | acid (g) | acid (%) | acid (%) |
| Example 2 | 5.33 | 95 | 99.3 |
| Example 3 | 4.77 | 85 | 99.6 |
| Example 4 | 5.39 | 96 | 99.5 |

According to the catalytic reduction method as described in Example 4, the Fe₃O₄@NC/Pt recovered in Example 4 was recycled for a catalytic reduction. The yield of 2-amino-5-fluorobenzoic acid in the cycle experiment and the recovery rate of the core-shell structure supported catalyst are listed in Table 2.

TABLE 2
The yield of 2-amino-5-fluorobenzoic acid in the cycle
experiment and the recovery rate of the Fe₃O₄@NC/Pt

| | Yield of 2-amino- | |
|---|---|---|
| Cycle number | 5-fluorobenzoic | Recovery rate of the |
| of catalyst | acid (%) | Fe₃O₄@NC/Pt (%) |
| The first | 96 | 99 |
| The second | 96 | 99 |
| The third | 96 | 98 |
| The fourth | 95 | 98 |
| The fifth | 95 | 96 |
| The sixth | 93 | 96 |
| The seventh | 93 | 95 |
| The eighth | 92 | 94 |
| The ninth | 91 | 92 |
| The tenth | 90 | 92 |

From the data in Table 2, it can be seen that the core-shell structure supported catalyst provided by the present disclosure can be recovered by magnetic separation, and the recovery rate is 93-99%. The recovered core-shell structure supported catalyst can be recycled and reused, and the yield of 2-amino-5-fluorobenzoic acid prepared by the catalytic reduction is 90-96%.

Although the above embodiments give a detailed description of the present disclosure, they are only a part of the embodiments of the present disclosure, rather than all the embodiments. Those skilled in the art could also obtain other embodiments based on above embodiments without creativity. These embodiments all fall within the protection scope of the present disclosure.

What is claimed is:

1. A core-shell structure supported catalyst, comprising a core-shell structure carrier and platinum supported on a surface of the core-shell structure carrier, wherein
the core-shell structure carrier comprises a ferroferric oxide nanoparticle core and a nitrogen-doped carbon shell, and
a molar ratio of the ferroferric oxide nanoparticle core to platinum is in the range of 1:(0.03-0.3).

2. The core-shell structure supported catalyst of claim 1, wherein the core-shell structure supported catalyst has a particle size of 20-100 nm.

3. The core-shell structure supported catalyst of claim 1, wherein a mass ratio of the ferroferric oxide nanoparticle core to the nitrogen-doped carbon shell is in the range of (50-200):1;
a mass percentage of carbon in the nitrogen-doped carbon shell is in the range of 0.1-10%;
a mass percentage of nitrogen in the nitrogen-doped carbon shell is in the range of 0.02-5%.

4. A method for preparing the core-shell structure supported catalyst of claim 1, comprising
dispersing ferroferric oxide nanoparticles and chitosan in a first solvent, to obtain a dispersed mixture, and subjecting the dispersed mixture to a carbonization reaction, to obtain a core-shell structure carrier; and
mixing the core-shell structure carrier, a platinum compound, a reducing agent and a second solvent, to obtain a mixed solution, and subjecting the mixed solution to a reduction reaction, to obtain the core-shell structure supported catalyst.

5. The method of claim 4, wherein the platinum compound is one selected from the group consisting of platinum chloride, chloroplatinic acid hexahydrate, and sodium hexachloroplatinate;
the reducing agent is one selected from the group consisting of ascorbic acid, hydrazine hydrate, and sodium borohydride.

6. The method of claim 4, wherein the first solvent is an aqueous acetic acid solution;
the carbonization reaction is performed at a temperature of 180-200° C. for 10-12 h.

7. The method of claim 4, wherein the second solvent is one selected from the group consisting of an aqueous ethanol solution, water or an aqueous methanol solution;
the reduction reaction is performed at a temperature of 60-100° C. for 2-3 h.

8. The method of claim 4, wherein the ferroferric oxide nanoparticles are prepared by a method comprising:
mixing an iron compound, ethylene glycol, polyvinylpyrrolidone and sodium acetate, to obtain a mixture and subjecting the mixture to a solvothermal reaction, to obtain the ferroferric oxide nanoparticle.

9. A method of using the core-shell structure supported catalyst of claim 1, comprising using the core-shell structure supported catalyst in the preparation of 2-amino-5-fluorobenzoic acid.

10. The method of claim 9, wherein the preparation of 2-amino-5-fluorobenzoic acid comprises:
mixing 5-fluoro-2-nitrobenzoic acid, water and the core-shell structure supported catalyst, to obtain a reaction solution, wherein a mass ratio of 5-fluoro-2-nitrobenzoic acid to the core-shell structure supported catalyst is in the range of (5-20):1; and
adding ammonium formate in the reaction solution to obtain a solution mixture, and subjecting the solution mixture to a reduction reaction, to obtain 2-amino-5-fluorobenzoic acid.

11. The method of claim 4, wherein the core-shell structure supported catalyst has a particle size of 20-100 nm.
